(19) United States
(12) Patent Application Publication
(10) Pub. No.: US 2026/0053873 A1
(43) Pub. Date: Feb. 26, 2026

(54) COMBINATION THERAPY OF ONCOLYTIC VIRUS DRUGS FOR CANCER TREATMENT

(71) Applicant: SICHUAN ONCOCARE BIOPHARMACEUTICAL, INC., Chengdu (CN)

(72) Inventor: Li YU, Chengdu (CN)

(21) Appl. No.: 19/252,438

(22) Filed: Jun. 27, 2025

Related U.S. Application Data
(63) Continuation-in-part of application No. 16/752,328, filed on Jan. 24, 2020, now abandoned.
Application No. 16/752,328 is a continuation of application No. PCT/CN2019/098798, filed on Aug. 1, 2019.

(30) Foreign Application Priority Data
Jan. 7, 2019 (CN) 201910012374.8

(51) Int. Cl. A61K 35/768; A61P 35/00; A61P 37/04; C07K 14/705; C12N 7/00

(52) U.S. Cl. CPC A61K 35/768; A61P 35/00; A61P 37/04; C07K 14/70532; C12N 7/00; C12N 2770/24122; C12N 2770/24132; C12N 2770/24143; C12N 2770/24162; C12N 2770/24171

(57) ABSTRACT

A combination therapeutic strategy employs multiple oncolytic viruses (OVs), grouped and administered based on their virological properties, tumor selectivity, and distinct antigen profiles, to treat malignant tumors. Representative groupings may include members of flaviviruses, each contributing distinct immune modulation and the same modes of tumor cell killing. A predefined treatment schedule involves sequential or concurrent administration of antigenically diverse OVs in multiple cycles, reducing cross-neutralization and sustaining cytolytic pressure on tumors. A pre-characterized OV panel, comprising members from a virus family, RNA and DNA viruses, both wild-type and genetically engineered, serves as a flexible resource for customizing treatment regimens by tumor type, immune landscape, and therapeutic goals. This platform establishes a rational framework for combination OV therapy with improved durability, safety, and clinical effectiveness.

### CROSS-REFERENCE TO RELATED APPLICATION

This application is a continuation-in-part of U.S. patent application Ser. No. 16/752,328, filed on Jan. 24, 2020, which claims the priority to International Application No. PCT/CN 2019/098798, filed on Aug. 1, 2019, which claims priority to Chinese Patent Application No. 201910012374.8 filed on Jan. 7, 2019, the entire contents of all of which are incorporated herein by reference.

### TECHNICAL FIELD

The present disclosure relates to the field of biopharmaceutical and oncolytic cancer therapy.

### BACKGROUND

### Current Cancer Statistics and the Unmet Need For Effective Therapies

Cancer continues to be a major global health challenge. In 2022, the World Health Organization (WHO) reported approximately 20 million new cancer cases and 9.7 million cancer-related deaths worldwide. This translates to one in five people being diagnosed with cancer and one in nine men or one in twelve women dying from cancer during their lifetime.

By 2023, the American Cancer Society estimated around 1.9 million new cancer cases and 609,820 cancer deaths in the U.S. Globally, new cancer cases are projected to rise to 35 million annually by 2050, driven largely by population growth and aging. Strong inequities persist, as low- and middle-income countries continue to bear a disproportionately high burden, with cervical and liver cancers highly prevalent.

Despite improved survival rates in many cancers, such as a one-third decline in U.S. cancer deaths since 1991 and childhood cancer 5-year survival rates rising from 49% in the 1970s to ˜69% by 2019, the absolute number of cancer deaths remains high, and current therapies still fall short for many cancers and geographies.

### Oncolytic Virus (OV) Therapy: A Promising Yet Challenged Strategy

Oncolytic viruses (OVs) offer a dual mechanism of action: selective replication in tumor cells leading to oncolysis, plus stimulation of antitumor immunity as viral particles and tumor antigens prime host responses.

As of early 2024, 188 OV-related clinical trials are registered on ClinicalTrials. gov. These include: Phase I: ˜99 trials; Phase II: ˜35 trials; Phase III: 4 trials. Indications span melanoma, glioblastoma, breast, lung, colorectal, pancreatic, liver, bladder, and other cancers. Despite over 100 OV candidates in trials, only three OV therapies are approved globally: IMLYGIC (T-VEC), an HSV-1 engineered to express GM CSF, FDA-approved in 2015 for unresectable melanoma; DELYTACT (G47Δ), a genetically modified HSV-1 approved in Japan (2021) for recurrent glioblastoma, demonstrating ˜84% one-year survival; Oncorine (H101), an E1B-deleted adenovirus approved in China since 2005 for nasopharyngeal cancers. Another OV, Adstiladrin, a non-OV gene therapy for bladder cancer, was FDA-approved in late 2022.

Nevertheless, many challenges persist: Most trials are early-stage (Phase I/II). Delivery remains a barrier; and intratumoral injection is commonly required to achieve localized effect.

### Unmet Need

There is a clear gap between the promise of OV therapy and its translation into widely effective, approved treatments. To address this unmet need, there is an urgent demand for OV platforms with better delivery, enhanced tumor selectivity, and stronger immune activation to cancers but less response to OV.

### Limitations of Oncolytic Virus Monotherapy

Despite their therapeutic promise, several limitations hinder the widespread application and sustained efficacy of oncolytic virus therapy. As immunogenic biological agents, OVs are rapidly recognized by the host immune system. The presence of pre-existing immunity or the development of antiviral responses during treatment can lead to the production of neutralizing antibodies and cytotoxic T lymphocyte (CTL) activity, which restrict viral replication and dissemination. This is analogous to the mechanism by which vaccines protect against viral infections. In clinical settings, such immune responses significantly limit the duration and magnitude of OV efficacy, particularly when a single virus strain is used repeatedly.

In addition to immune clearance, the tropism of oncolytic viruses, defined by their affinity for infecting specific tissues or cell types, represents another significant challenge. Viral tropism is influenced by two main factors: (1) the physical accessibility of the virus to the target tissue and (2) the permissiveness of the host cell to support viral replication. As a result, many oncolytic viruses exhibit a limited range of infectivity, restricting their use to specific tumor types.

These immunological and virological hurdles help explain the relatively modest clinical success rate of monotherapy OV treatments.

### Rationale for Combination Oncolytic Virus Therapy

To overcome the limitations associated with single-agent OV therapy, one key strategy is to circumvent the host immune response by employing viruses with little or no pre-existing immunity in the patient population. A rational approach is to utilize a combination of oncolytic viruses possessing distinct antigenic profiles. By administering different serotypes of viruses, the likelihood of immune-mediated resistance against a single virus strain can be significantly reduced, allowing for prolonged and more effective tumor targeting therapy.

For example: The Flavivirus genus includes multiple serotypes with diverse serostatus, epidemiological and vaccination backgrounds. Leveraging this diversity, a series of oncolytic flaviviruses were developed, each of which can be selectively deployed in geographic regions where prior exposure, vaccination, or epidemic history is minimal or absent. This selective administration enables effective viral replication and tumor targeting with minimal interference from host immunity. Moreover, two or more distinct flavivirus serotypes can be sequentially or simultaneously combined into a multi-cycle treatment regimen. This strategy reduces the immunological limitations posed by repeated use of a single virus. By incorporating multiple attenuated or engineered OV strains, the therapy benefits from diversified mechanisms of action, broader tumor coverage, and a reduced likelihood of OV immunogenicity.

Additionally, owing to similar genetics and biological properties, different serotypes or antigen profiles of flavivirus members may have the same anticancer spectrum. Therefore, when they are grouped in a treatment regimen, they express consistency to cancer targeting or tropism and play the similar tumor-killing effect. Therefore, sequential administration of the combined OVs enhances therapeutic efficacy, and thus provides an adaptable approach to advancing oncolytic virus therapy in the fight against cancer.

### SUMMARY

The present disclosure provides a therapy strategy that combines different serotypes of oncolytic viruses as a drug group for cancer treatment.

Optionally, the combined oncolytic virus drugs may come from two or more members of a virus family, and may also be selected from different genus of oncolytic viruses with the same cancer spectrum.

Optionally, the multiple oncolytic virus strains can be selectively combined to form one or more groups in a treatment regimen. The combined oncolytic virus drugs can be sequentially and alternately administrated in treatment course to void drug resistance caused by viral antigenicity during monotherapy, and thus to meet the durable therapy needs.

Optionally, since there are multiple DNA and RNA oncolytic viruses are available, they can constitute a different candidate drug panel/pool according to their similar anticancer spectrum, and thus can be selectively grouped to treat specifically targeting cancers.

Optionally, the possibly combined oncolytic viruses include positive-and negative-sense RNA viruses attenuated or mutated to be less pathogenesis, between RNA and DNA viruses, as well as DNA viruses with safe records.

Furthermore, optionally, the grouped oncolytic viruses may include native viruses or artificial engineering viruses with or without inserting foreign genes.

### BRIEF DESCRIPTION OF THE DRAWINGS

The following drawings are merely examples for illustrative purposes according to various embodiments of the present disclosure and are not intended to limit the scope of the present disclosure.

FIG. 1 illustrates a structural diagram of a recombinant oncolytic flavivirus, according to some embodiments.

FIG. 2 illustrates an indirect fluorescent antibody (IFA) detection of oncolytic West Nile virus (WNV) replication and foreign gene expression, according to some embodiments.

FIG. 3 illustrates cancer cell cytopathic effect (CPE) caused by oncolytic WNV, according to some embodiments.

FIG. 4 illustrates oncolytic WNV repression of mouse tumor growth, according to some embodiments.

FIG. 5 illustrates oncolytic WNV, YFV, and JEV that share the same infectivity to specific cancer cell lines, according to some embodiments.

FIG. 6 illustrates a combination therapy with three oncolytic OVs, according to some embodiments.

### DETAILED DESCRIPTION OF THE PREFERRED EMBODIMENT

The described embodiments of the present disclosure relate to a combination therapy strategy employing multiple oncolytic viruses grouped according to their virological properties and anticancer activity spectrum to effectively treat malignant tumors. This combinatorial approach is designed to enhance therapeutic efficacy, broaden tumor targeting, and minimize the development of antiviral resistance commonly seen with monotherapy OV regimens.

### 1. General Design of the Combination Therapy

In the disclosed embodiment, a treatment course for cancer is established using a panel of two or more distinct oncolytic viruses, administered in a scheduled, rotational, or simultaneous manner. The viruses are grouped based on shared or complementary tumor cell tropism, mechanism of oncolysis, or immune activation properties. The design of the group may also consider the serotype, family, or genus classification of the viruses to diversify antigenic profiles, reduce cross-immunity, and maintain tumor selectivity over the course of therapy.

### 2. Selection Criteria for Oncolytic Viruses

In one embodiment, the combination group is assembled from viruses belonging to: either a same virus family but different serotypes (e.g., different coxsackievirus serotypes within the Picornaviridae family or different serotype members within flavivirus genus); or different viral genera or families (e.g., Paramyxoviridae, Rhabdoviridae, Adenoviridae, Poxviridae, Flaviviridae, or Reoviridae) with overlapping tumor-killing spectra.

The viruses can be selected based on but not limited to the following characteristics: demonstrated or engineered tumor-selective replication, known oncolytic potency and immunogenic cell death (ICD) profile, low or manageable pathogenicity in humans, non-overlapping host immunity to minimize cross-neutralization, and compatibility with different routes of administration (e.g., intravenous, intratumoral, intraperitoneal, or inhalational delivery).

### 3. Representative Group Composition

In one embodiment, a treatment group consists of three viruses: an engineered vesicular stomatitis virus (VSV) lacking the matrix (M) protein, to reduce neurovirulence while preserving oncolytic activity; a serotype B adenovirus (Ad5) expressing a tumor-specific promoter-driven lytic gene; and a mutated measles virus (MV-Edm strain) with engineered immune factors (such as CD46 targeting and IFN-γ expression).

This group is designed to target solid tumors such as pancreatic, colorectal, and hepatocellular carcinoma, all of which share susceptibility to the above agents. Each virus can be administered sequentially or in overlap, with periodic assessment of immune markers and tumor burden to determine transition points (i.e., the time points to switch OV or OV combination) in the course.

### 4. Candidate Pooling Strategy

In some embodiments, to optimize flexibility in treatment design, a candidate OV panel is pre-characterized for: antitumor spectrum, oncolytic mechanism (e.g., direct lysis, immune modulation, anti-angiogenesis, etc.), and antigenic features and patient immune landscape.

In some embodiments, the panel can include both RNA viruses (e.g., reovirus, VSV, measles virus, Seneca Valley virus, and flavivirus derivatives) and DNA viruses (e.g., adenovirus, herpes simplex virus, vaccinia virus). Some viruses may be wild-type isolates, while others are genetically engineered to: enhance selectivity (e.g., via promoter control or tropism modification), insert immune genes (e.g., GM-CSF, IL-12, PD-L1 inhibitors, T-cell co-stimulators), and/or improve replication or persistence in tumor environments.

The selected OVs are grouped into modular treatment sets, based on similar tissue tropism or immune interaction profiles. Each set may be rotated in multi-cycle treatment schedules to prevent immune-mediated resistance and prolong efficacy.

### 5. Resistance Avoidance and Sequential Therapy

The combination therapy specifically addresses the risk of anti-viral neutralizing antibody development, a key limitation in repeated oncolytic virus administration, by rotating or co-administering antigenically distinct viruses. In this way, the host immune system is less likely to neutralize all agents simultaneously, thus preserving the cytolytic pressure on tumor cells. In a typical embodiment:

Cycle 1: Administer OV-A (e.g., measles virus) via intravenous route;

Cycle 2: Administer OV-B (e.g., VSV) via intratumoral injection; and

Cycle 3: Administer OV-C (e.g., adenovirus) via peritumoral injection or aerosol (for lung lesions).

Each cycle may be 7-14 days in length, repeated up to 4-6 cycles depending on tumor response and toxicity.

### 6. Therapeutic Indications

In one embodiments, the combination therapy is designed for solid tumors and hematologic malignancies, particularly: cancers with poor response to standard chemotherapy (e.g., glioblastoma, pancreatic adenocarcinoma), tumors with low mutational burden or poor immunogenicity (often called “cold tumors”, such as pancreatic ductal adenocarcinoma (PDAC), prostate cancer, glioblastoma (GBM), ovarian cancer (especially low-grade serous subtype), liver cancer (hepatocellular carcinoma, HCC), etc.), where poly-OV therapy can convert “cold” tumors to “hot” immune microenvironments, recurrent or metastatic cancers in immune-competent patients where virus neutralization would otherwise limit single-agent efficacy.

### 7. Optional Genetic Modifications

Each virus in the group may optionally carry: tumor-specific promoters for selective gene expression, suicide genes for safety off-switches (e.g., TK/GCV systems), and antigen expression cassettes to prime anti-tumor immunity or serve as neoantigen vaccines.

These modifications can be harmonized across the virus group to create synergistic immuno-oncologic effects.

The present disclosure aims to overcome the defects of the existing oncolytic virus treatment and provide novel pharmaceutics for cancer therapy.

Specifical example: The application of a combination of flavivirus-oncolytic drugs can reduce the immune resistance to a single oncolytic virus drug and thus effectively treat cancers.

The present disclosure elaborates a series of oncolytic flavivirus drugs that may enhance cancer treatment when combined with administering as a group drug. The active ingredients of medicines comprise positive single-stranded-RNA viruses with or without foreign gene fragments that specifically induce an immune response against cancers. The application of a combination of flavivirus-oncolytic drugs can avoid inhibiting oncolytic virus by the preformed immune defense and can reduce immune resistance for more rounds of administration, thus enhancing the efficacy of cancer therapy.

The flavivirus genus consists of more than 70 small, positive-sense, single-stranded RNA viruses transmitted by arthropods, particularly mosquitoes and ticks. Forty species of flavivirus members may be associated with human diseases. These include globally critical human pathogens such as West Nile virus (WNV), Japanese encephalitis virus (JEV), dengue virus (DENV), Murray Valley encephalitis virus (MVE), tick-borne encephalitis virus (TBEV), Yellow Fever virus (YFV), and Zika virus (ZIKV).

Flaviviruses are small enveloped viruses (approximately 50 nm in diameter). Their genome of ˜11 kb contains a single open reading frame (ORF) flanked by untranslated regions and encodes three structural proteins and seven non-structural proteins. The ORF is flanked by a 5′ noncoding region (NCR), which is about 100 nucleotides (nt), and by a 3′-NCR, which is 400 to 800 nucleotides. The mature virion features a surface densely covered with E glycoproteins and M proteins and a core consisting of capsid (C) protein and the RNA genome.

Flaviviruses can replicate in various species and have a broad cellular tropism, including epithelial cells. They also infect many cell lines in vitro. The flavivirus envelope protein is the dominant antigen for eliciting neutralizing antibodies and plays an important role in inducing immunologic responses in the infected host. Each flavivirus ′s E protein possesses a different antigen phenotype because its amino acid composition is various.

Currently, approved available flavivirus vaccines include yellow fever (17D), Japanese encephalitis (SA14-14-2), Dengue viruses, attenuated West Nile, and tick-borne encephalitis. These vaccine strains plus chimeric viruses may comprise a panel/bank of oncolytic flaviviruses with different serotypes (antigens). The panel provides a choice to choose an adequate serotype strain to administer to patients in a particular area. It can avoid repeatedly using a single serotype strain when requiring multiple treatments. Different flaviviruses can be selectively used (A) in the non-vaccination regions and (B) alternately administrated in a treatment course. Therefore, the application of multiple oncolytic flaviviruses as a group in OV therapy significantly reduces immune pressure to single OV therapy. The combination but alternate administration may produce maximal efficiency in the cancer therapy.

For example, using oncolytic WNV (attenuated strain) instead of JEV to treat cancer may be a practical and effective way to avoid immune resistance to JEV, to which the population has a high immunity owing to broad vaccination (e.g., in China). Applying an oncolytic JEV vaccine strain in Northern America would be an excellent alternative to an oncolytic yellow fever strain. In addition, with multiple attenuated virus strains, more options may be available in designing a treatment plan.

Formulating several oncolytic viruses with distinct antigens in a treatment course can avoid systemic immunity to single OV inhibition and thus effectively enhance cancer treatment.

As an oncolytic virus, the flavivirus genus is positioned to be an excellent drug candidate owing to the multiple available stains. Alternate application or combination of numerous attenuated strains can increase efficacy because of no cross-reaction among their serotypes, thus avoiding preformed immunity to virus antigens. Each Flavivirus has a similar gene structure and protein profile and a genome of a smaller size, therefore making it easy to manipulate and integrate therapeutic genes. They have broad tropism to mainly infect epithelial cells, which occur in 85% of cancers. Furthermore, RNA oncolytic virus drugs have more advantages than DNA oncolytic viruses: they have fewer viral proteins, and the viral genes do not integrate into the host chromosome to cause oncogenic mutation and latent infection.

We have constructed oncolytic flavivirus vectors that carry a variety of T cell costimulators and lymphocyte factors, respectively. These oncolytic flaviviruses have also been tested in mouse tumor models and showed very promising results. Eighty percent repression of tumor growth was observed in two tumors of a mouse with only one intratumor injection, reflecting a systemic immune response to the tumor that was not injected with a drug.

### Example 1

### Generation of Attenuated Flaviviruses

Considering the safety of using viruses as human drugs, it must be proven that these flaviviruses have been attenuated and will not cause human disease. In fact, many attenuated flaviviruses have been used as vaccines against human flavivirus infections.

### Attenuation of WNV

Point mutation of WNV E protein resulted in neuro-attenuated flaviviruses. Applicant modified the infectious WNV cDNA and replaced five neuron-related amino acids on the WNV envelope, including:

(i) The 138^{th} Glutamic acid E was replaced with Lysine K;

(ii) The 172^{nd} Tyrosine Y was replaced with Valine V;

(iii) The 173^{rd} Threonine T was replaced with Alanine A;

(iv) The 276^{th} Lysine K was replaced with Methionine M; and

(v) The 312^{th} Alanine A was replaced with Valine V.

The resulting mutated WNV (named “WV/Env5”), when transformed, showed attenuating toxicity. When the WNV envelope mutant (WN/Env5) was injected into the brain of baby mice, they showed a 1000-fold reduction in neurotoxicity. When the WN/Env5 was used to infect the mice through subcutaneous or intravenous injection, no mice died, demonstrating a successful attenuation of WNV with E mutation. The attenuated WNV is thereby prevented from infecting the CNS through the blood-brain barrier. While it loses its ability to infect the CNS from the periphery, it can still replicate and kill tumor cells and effectively suppress tumor growth in humans and mice as demonstrated by Investigator Initiated Test in human and preclinical animal studies. Thus, the attenuated WNV is an ideal OV candidate and can be also used as a part of the combination therapy disclosed herein.

### Example 2

### Construction of Infectious Flaviviruses Carrying Foreign Gene Fragments

In some embodiments, a mutated oncolytic virus disclosed herein may further contain artificially inserted exogenous gene fragments. These exogenous gene fragments are mainly active immune agents, including but are not limited to human T cell costimulators. These non-viral exogenous gene fragments are inserted into different types of flavivirus genomes using conventional and commonly adopted genetic engineering methods.

### WNV Virus Carrying T Cell Costimulatory Gene

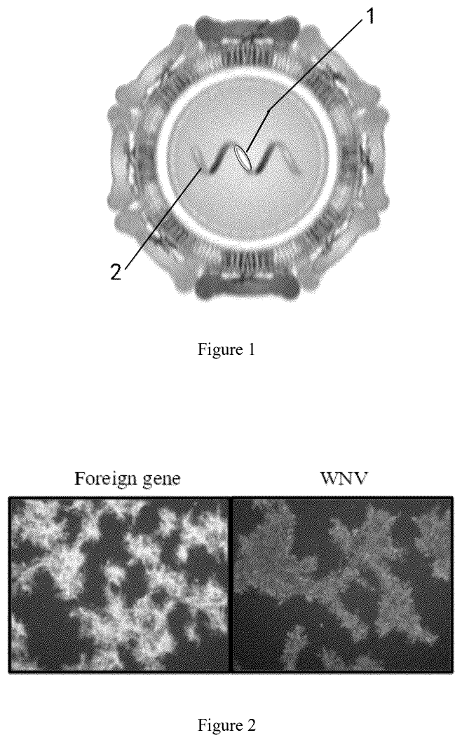

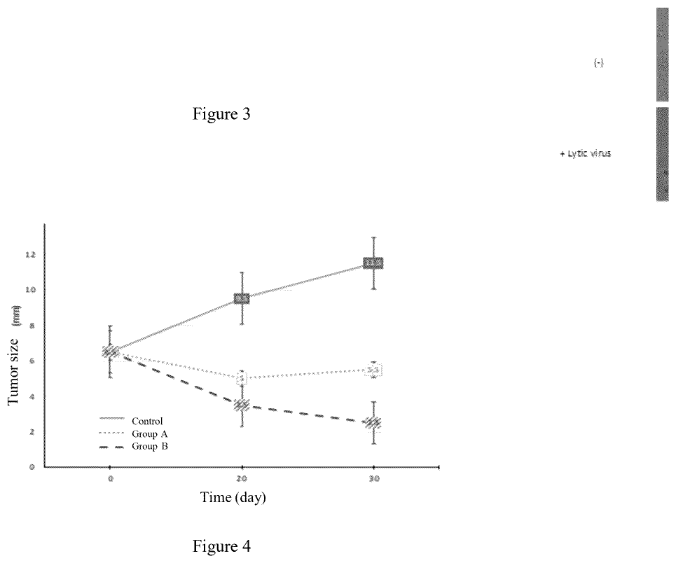

The attenuated WNV described in Example 1 were genetically modified to contain a T cell costimulator gene segment. The conventional methods for engineering WNV vectors include: PCR synthesizes human or mouse T cell gene (CD86) fragments; ligate these fragments to the attenuated WNV cDNA through restriction enzyme sites (NheI and AgeI, artificially created) between the E and NS1 genes; and colony selection of recombinants through restriction enzyme mapping and sequencing. The cloned recombinant plasmids were further examined for virus infection and foreign gene expression in cultured cells in vitro. As shown in FIG. 2, the immunofluorescence assay detected the WNV replication and the expression of the T cell costimulator. When lung or cervical cancer tumor cells were infected with these recombinant flaviviruses, the cells died from oncolytic virus infection after three days. The virus was genetically stable without losing the foreign gene expression after the 10^{th} consecutive passage in Vero cells. These results indicate that the insertion of foreign genes did not affect recombinant flavivirus replication.

Sensitive mouse experiments did not reveal neuroinvasive disease or mouse death. These data prove that recombinant flaviviruses do not increase variability but reduce toxicity (pathogenicity) as expected. The virus was genetically stable during the 20^{th} consecutive passage in Vero cells, as demonstrated by IFA tests. Thus, they are safer for clinical applications.

### Example 3

### Evaluation of Oncolytic Virus in Mouse Tumor Models

A mouse stomach tumor (MFC) model was established. Briefly, mouse MFC tumor cells were cultured in RPMI1640 medium containing 5% fetal bovine serum. MFC cells (5 million) growing at the logarithmic growth stage were then injected subcutaneously at two sides of the dorsal of 6-8-week-old mice (C57BL/6) to establish the MFC model mice. When MFC tumors on the dorsal grow to an average diameter of 5-6.5 mm after about eight days, the DNA form of recombinant WNV was injected into the right-side tumor only.

### Animal Groups

1. Control group: 100 μof PBS.

2. Experimental group A: 100 μg/100 μl WE/Hc86-(WNV virus carries human B7 gene fragment).

3. Experimental group B: 100 μg/100 μl WE/Mc86-(WNV virus carries mouse B7 gene fragment).

The experimental results (shown in FIG. 4) are as follows:

1. In all 9 test mice, no symptoms were observed within 10 days after injection, and no death occurred within 30 days.

2. 20 days after the inoculation, the diameter of the tumor was measured: the average tumor diameter of the control group was 9.5 mm, the experimental group A was 5.0 mm, and the experimental group B was 3.5 mm.

3. 30 days after inoculation, the diameter of the tumor was measured: the average tumor diameter in the control group was 11.5 mm, the experimental group A was 5.5 mm, and the experimental group B was 2.5 mm.

4. Thirty days after the inoculation, tumor tissue sections and histochemical examination showed that there was much T cell infiltration around the residual tumor in the experimental group B, which was several times more than the experimental group A.

### CONCLUSION

1. Inhibition of tumor growth was significantly different between groups. Compared with the control group, the tumor growth of the experimental group A was significantly suppressed, while the tumor of the experimental group B almost disappeared.

2. The difference in the number of T cell infiltration may be attributed to the activation response of the experimental group B mouse-derived B7 to the immune system.

3. WNV oncolytic virus carrying foreign gene fragments is safe as a therapeutic drug candidate.

### Example 4

Flavivirus OVs share the same profile of anticancer cells.

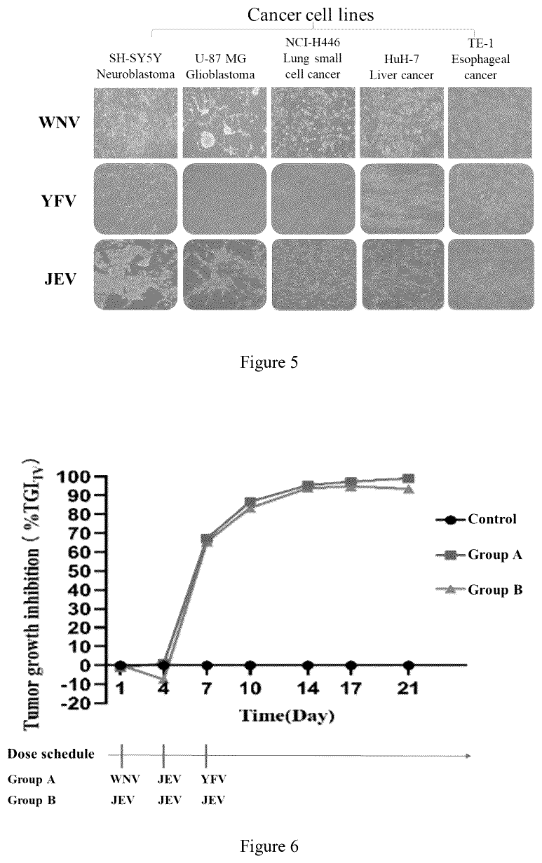

More than 50 cancer cell lines were screened to determine which cancer cells would be susceptible to OV infection. Surprisingly, less than 10 could be infected by the flavivirus OV. However, 5 of the 10 susceptible cell lines, SH-SY5Y-neuroblastoma, U-87-MG Glioblastoma, NCI-H446-lung small cell cancer, HuH7-liver cancer, and TE1-esophageal squamous carcinoma, were commonly sensitive to WNV, YFV, and JEV infection, as can be seen from FIG. 5. These results indicate that these OVs have the same cancer indications and thus provide the basis for the combination of these OVs as a group in treating these cancers. In other words, these results suggest that WNV, JEV, and YFV share a common cancer tropism, and thus they can infect and potentially kill the same types of cancer cells. This overlapping infectivity spectrum supports the rationale for combining these viruses in a sequential or rotational OV therapy strategy. Since these viruses are antigenically distinct but functionally similar, they can enhance therapeutic durability while avoiding immune resistance due to serotype switching.

### Example 5

The inhibition effects on mouse tumor by a combination treatment with three flavivirus OV.

A mouse neuroblastoma subcutaneous tumor model was established by dorsal subcutaneous injection of mouse neuroblastoma cells (Neuro-2a) in 6-8-week-old mice (A/J GPT). When the dorsal tumor grew to an average diameter of 5.5-6.5 mm for about 7 days, the three oncolytic viruses WNV, JEV, and YFV were administered to the tumor separately in three-day interval sequentially.

### Animal Groups

1. Control group: 0.9% NaCl.

2. Experimental group A: Combination of WNV 10⁶PFU, JEV 10⁶PFU, YFV 10⁵PFU.

3. Experimental group B: JEV 10⁶PFU/mouse

The experimental results are as follows:

1. In all 18 test mice, no symptoms were observed within 21 days after administration.

2. There was no significant difference in the tumor volume of mice in the control group and experimental groups A and B on Day 1 to Day 4 after injection (p [symbol] 0.05).

3. On Day 7, after the third treatment, the average tumor volume of mice in the control group was 327.66±38.90 mm³, and the average tumor volumes of mice in groups A and B were 107.24±15.39 mm³ and 112.94±14.21 mm³, respectively. On Day 21, the average tumor volume of mice in the control group was 3469.92±493.64 mm³, and the average tumor volumes of mice in group A and group B were 29.04±7.48 mm³ and 225.66±176.81 mm³, respectively.

### CONCLUSION

Both combination therapy with WNV/JEV/YFV and JEV monotherapy can effectively inhibit the growth and proliferation of mouse neuroblastoma (Neuro-2a) in A/J mice. The percent tumor growth inhibition of tumor volume (%TGITV) of the experimental group A was 99.16%, and the %TGITV of experimental group B was 93.50%. The tumor inhibition rate in group A (combination therapy) was slightly higher than that of group B (single therapy), as shown in FIG. 6. This supports the core idea of the present disclosure: combination therapy reduces immune resistance and improves tumor control more effectively than monotherapy.

What is claimed is:

1. A method for treating cancer in a patient in need thereof, comprising administering a combination of two or more oncolytic viruses selected from different serotypes, species, genera, or families, wherein the oncolytic viruses are selected based on having similar or complementary anticancer activity spectra and non-overlapping immunogenic profiles, thereby reducing antiviral immune resistance and enhancing therapeutic efficacy.

2. The method of claim 1, wherein the oncolytic viruses are administered in a sequential or alternating regimen during a treatment course.

3. The method of claim 1, wherein at least one of the oncolytic viruses is selected from Flavivirus genus, comprising different serotypes with distinct vaccination or epidemic histories in a treatment population.

4. The method of claim 1, wherein the oncolytic viruses are selected from both RNA and DNA viruses.

5. The method of claim 4, wherein the RNA viruses comprise one or more of the following: vesicular stomatitis virus (VSV), measles virus (MV), reovirus, Seneca Valley virus (SVV), or attenuated flavivirus.

6. The method of claim 4, wherein the DNA viruses comprise one or more of the following: adenovirus, herpes simplex virus (HSV), or vaccinia virus.

7. The method of claim 3, wherein at least one of the selected Flavivirus genus oncolytic viruses is an attenuated WNV containing point mutations within E protein and containing a CD86 T-cell costimulator.

8. The method of claim 1, wherein one of the oncolytic viruses is genetically engineered to express an immune modulatory factor or a checkpoint inhibitor antagonist.

9. The method of claim 1, wherein the oncolytic viruses are administered through different routes selected from intratumoral, intravenous, intraperitoneal, or inhalational delivery.

10. The method of claim 1, wherein each oncolytic virus is formulated for administration as a separate dose and combined treatment regimen.

11. The method of claim 2, wherein the treatment course comprises over one cycle, each employing at least one different oncolytic virus.

12. The method of claim 1, wherein a selection of an oncolytic virus to be included in the combination is determined based on patient serostatus or epidemiological data indicating low pre-existing immunity against one or more of the oncolytic viruses.

13. The method of claim 1, wherein the combination of two or more oncolytic viruses target epithelial-derived cancers comprising more than 80% of solid tumor types.

14. The method of claim 1, wherein oncolytic virus replication is driven by tumor-specific promoters to enhance tumor selectivity.

15. The method of claim 1, wherein at least one oncolytic virus is genetically engineered to express a tumor-associated antigen for in situ vaccination.

16. The method of claim 1, wherein the treatment is applied to tumors with low mutational burden or poor immunogenicity.

17. A pharmaceutical composition comprising two or more oncolytic viruses selected from different serotypes or virus genera, wherein the oncolytic viruses are formulated for sequential administration in a multi-stage cancer treatment protocol.

18. The pharmaceutical composition of claim 17, wherein an oncolytic virus is an attenuated or mutationally engineered.

19. The pharmaceutical composition of claim 17, wherein one of the oncolytic viruses is genetically engineered to express an immune modulatory factor or a checkpoint inhibitor antagonist.

20. The pharmaceutical composition of claim 17, wherein a selection of an oncolytic virus to be in the combination is determined based on patient serostatus or epidemiological data indicating low pre-existing immunity against one or more of the oncolytic viruses.
